(19) United States
(12) Patent Application Publication
(10) Pub. No.: US 2010/0113366 A1
(43) Pub. Date: May 6, 2010

(54) USE OF PROLINE-RICH PEPTIDE DERIVED FROM HYPOTHALAMUS FOR THE MANUFACTURE OF A MEDICAMENT FOR THE TREATMENT AND/OR PROPHYLAXIS OF CATTLE LEUCOSIS

(76) Inventors: Armen Galoyan, Yerevan (AM); Ashkhen Shirvayan, Yerevan (AM)

Correspondence Address: PAUL AND PAUL, 2000 MARKET STREET, SUITE 2900, PHILADELPHIA, PA, 19103, US

(21) Appl. No.: 11/993,521

(22) PCT Filed: May 29, 2006
(86) PCT No.: PCT/AM2006/000005

§ 371 Date: Jan. 20, 2010

(30) Foreign Application Priority Data
Jun. 24, 2005 (AM) P20050113

(51) Int. Cl. A61K 38/10; C07K 7/08; A61P 35/00

(52) U.S. Cl. 514/14; 530/326

(57) ABSTRACT

The invention concerns to the medicinal preparation containing peptides and may be used for the treatment and/or prophylaxis of cattle leucosis.

The peptide comprising Ala-Gly-Ala-Pro-Glu-Pro-Ala-Glu-Pro-Ala-Gln-Pro-Gly-Val-Tyr amino acids residues sequence is intravenously administered to the cattle with leucosis. Peptide is present in a concentration 1.57×10⁻⁴ g/ml of physiological solution and administered in dose of 4×10⁻³ ml/kg live weight of animals twice in 48h.

There is an increase of the effectiveness of the cure of the cattle with leucosis.

### TECHNICAL FIELD

The present invention relates to a new use of hypothalamic PRP-1 peptide for the treatment and/or prophylaxis of cattle leucosis.

### BACKGROUND ART

Human and animal leucosis is considered one of the most important contemporary problems of medical and veterinary sciences. However, there is no any successful developed treatment and prophylaxis means, which can keep the infected individual from the lethal outcome until now. Nowadays in veterinary medicine, the fight with cattle leucosis is carried out by removal of ill and infected animals. Recently, with the purpose of prophylaxis of cattle leucosis, the administration of immunomodulators including T- and B-activins in calves was proposed. It is administered in 20-30-day calves subcutaneously once per day during 3 days. It regulates the activity of cellular and humoral factors of immune system in physiological norm limits during 12 months [10].

According to this point of view, the discovery of new biologically active substances surely should be directed to as organism immunomodulatory function forming and activity increasing, which because of their influence will promote the hematopoiesis regulation as well as the future current leucosis exacerbation process stopping.

### DISCLOSURE OF INVENTION

The new immunomodulators family discovered by us could have a similar positive influence on the animals with leucosis. These immunomodulators are secreted by magnocellular cells in the paraventricular nucleus and the supraoptic nucleus of the hypothalamus and named Proline-rich Polypeptides (PRPs) [7,8]. The most studied among them is PRP-1, which comprises the following amino acid sequence: Ala-Gly-Ala-Pro-Glu-Pro-Ala-Glu-Pro-Ala-Gln-Pro-Gly-Val-Tyr. Our studies demonstrated that PRP-1 possesses a number of important properties as immunocompetent cells concentration (T and B-cells and macrophages) [9], participates in interleukins (TNF, IL-1, IL-6) expression mechanisms in fibroblasts, strong antimicrobial and antiviral effect, neurotrophic influence on GFAP biosynthesis in astrocytes in vitro [1,2]. PRP-1 has a direct suppressing effect on human malignant T-cells—the influence on the proliferation of so-called Jurkat cells [3,8]. PRP-1 in concentrations 1×10⁻¹⁰-1×10⁻⁶ M strongly suppresses the proliferation of these cells in vitro. It is demonstrated that PRP-1 completely restores the myelopoiesis in mice with cyclophosphamide-induced lymphocytopenia [9].

It is known that the quantity and content as well as single leucocytes subpopulations changes during leucosis depend on the relative increase of transformed B-cells in the bloodsteam. It is characterized by the diagnostic (homeostatic, functional, structural) peculiarities. On the base of the relative content changes of above-mentioned parameters during the leucosis development a decrease of the quantity of small lymphocytes possessing the higher reactivity and resistance and an increase the quantity of large and medium lymphocytes, pro-lymphocytes and lymphoblasts [6].

The technical problem of the present invention is a providing of the effective treatment and/or prophylaxis of cattle leucosisn by simultaneous increasing of the activity of the immune system and selective destruction of the malignant transformed lymphocytes. This problem solves by use of PRP-1 peptide.

From this point of view, the study of the effectiveness of PRP-1 influence on the hematological and cytomorphological indices in naturally infected cows with leucosis in vitro and in vivo is of interest. The study of the PRP-1 administration effectiveness in vivo in infected naturally cows with leucosis was carried out by us for the first time.

The results of our study demonstrated that because of hypothalamic cytokine PRP-1 influence (in vitro), the significant changes of the hematological and cytomorphological indices of cattle with leucosis have place, which was proved by the degenerative structural destruction of large, medium lymphocytes, lymphoblasts and pro-lymphocytes, as well as malignant cells [5]. We can conclude that the PRP-1 influence can be more effective in vivo.

The cows with leucosis were administered with the PRP-1 solution in concentration 1.57×10⁻⁴ g in 1 ml in a dose of 4×10⁻³ ml/kg live weight of animal twice in 48 h.

### BRIEF DESCRIPTION OF DRAWINGS

FIG. 1 shows the peripheral blood smear of the cows with leucosis before PRP-1 administration.

FIG. 2 shows the peripheral blood smear of the cows with leucosis before PRP-1 administration

FIG. 3 shows the peripheral blood smear of the cows with leucosis in 48 h after 1^{st} PRP-1 administration

FIG. 4 shows the peripheral blood smear of the cows with leucosis in 48 h after 2^{nd} PRP-1 administration.

### BEST MODES FOR CARRYING OUT THE INVENTION

The blood samples were taken from the jugular vain of animal into the test tubes with anti-coagulant Trilon-B before administration, in 48 h after 1^{st} and 2^{nd} administrations, as well as in 33 days after the 2^{nd} administration. The blood hematomorphological and cytomorphological studies were carried out according to the available methods of veterinary hematology [4].

It is demonstrated that in 48 h after 1^{st} and 2^{nd} administration of PRP-1, a decrease of leucocytes by 24% (p<0.05) and 37% (p<0.01) the blood of the cows with leucosis was observed, as well as a drastic decrease of absolute and relative content of leucocytes by 34% and 43.4%, 14.6% and 21.3% (p<0.01 and p<0.001) accordingly. At the same time, the significant increase of hemoglobin (p<0.02) and erythrocytes content (p<0.01) was observed. Because of PRP-1 immunomodulatory influence in 48 h after 1^{st} and 2^{nd} administrations, a drastic (3-4 times) increase of the content of segmentonuclear neutrophils participating in the preliminary stages of phagocytosis (p<0.001) was observed.

A decrease of the relative content of large and medium lymphocytes with weak resistance in the blood of the cows with leucosis was found out in 48 h after 1^{st} and 2^{nd} administrations of PRP-1 by 11% and 10%, 23% and 30% accordingly. At the same time, an increase up to 60% and 59% of the relative content of small lymphocytes with strong resistance was shown. In 48 h after 1^{st} administration of PRP-1, we could not find any structurally atypical and hardly classifying lymphocytes in the blood smears, whereas their relative content was 2% before the administration. To find out the main point of the PRP-1 influence effectiveness, the subsequent study of the possible changes of the described identical blood parameters was done in 33 days after 2^{nd} PRP-1 administration. The obtained results convinced that the regulation process of hematological and cytomorphological indices in the organism of the cows with leucosis is continuing, and this data has a high reliability in comparison with the data obtained before the administration (p<0.01−p<0.001).

The results of the studies allow considering that thanks to the selective influence PRP-1, the regulation of hematopoiesis separate steps functions takes place, particularly erythroblastic and myeloblastic, as well as lymphoblastic, which are during the leucosis touched most of all. The literature data shows it also [9].

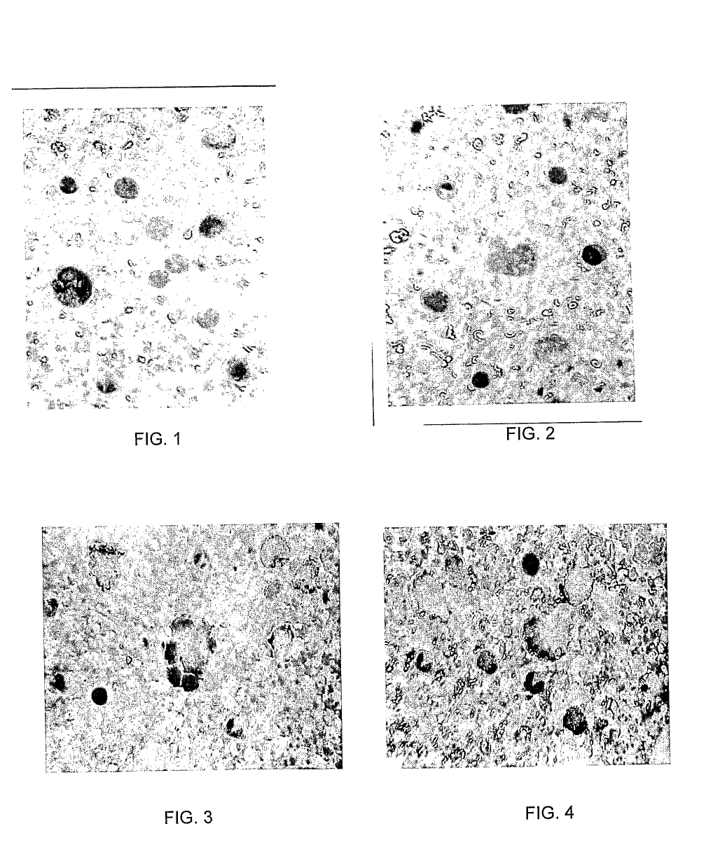

The microscopic analysis data demonstrated that before the PRP-1 administration, high quantities of lymphocytes are found (small, medium, large), rare prolymphocytes, as well as malignant cells (FIG. 1 and FIG. 2). In 48 h after 1^{st} and 2^{nd} administration of PRP-1 in the blood smears structurally destroyed and large and partially medium lymphocytes with weak resistance, prolymphocytes, lymphoblasts as well as malignant cells are found (FIG. 3 and FIG. 4). We should mention that no any morphological changes of small lymphocytes with high resistance, neutrophils (stab and segmentonuclear), eosinophils and monocytes were detected in the blood smears (FIG. 1-4).

In 33 days after PRP-1 administration, in the blood smears of the cows with leucosis the cytomorphological indices almost correspond to the indices of healthy cows, which are expressed by less differing and by the complete absence of malignant cells, the normalizing of separate lymphocytes subpopulations, as well as the absence of structural degradation processes of different types of lymphocytes.

### Conclusion

The research results are evidence that Proline-rich Polypeptide-1 manifests a significant suppressing effect on the malignant lymphocytes proliferation, regulates and restores changed hematopoietic organs functions. Along with this, PRP-1 thanks to its immunomodulatory properties, promotes the immune system functioning in animals with leucosis, as well as stop the further illness exacerbation.

### References

1. Aprikyan V. S., Galoyan A. A. Immunoprotective properties of a new hypothalamic polypeptide in bacterial pathologies//Med.Nauka Armenii-1999-Vol.39(2)-P.23-29.
2. Aprikyan V. S., Galoyan A. A. Immunocorrective properties of a new hypothalamic polypeptide in macrophage-associated bacterial dysfunctions//Med.Nauka Armenii-1999-Vol. 39(4)-P. 29-36.
3. Galoyan A. A., Shakhlamov V. A., Bogdanova I. M., Malaycev V. V., Mikhaileva L. M. A study of immunomodulatory properties of hypothalamic Proline-rich Polypeptide (PRP), in vitro//Neurokhimia (RAS and NAS RA)-2002-Vol.19(1)-P.41-45.
4. Simonyan G. A., Khisamutdinov F. F. Veterinary hematology//M. “Kolos”-1995-P.16-204.
5. Shirvanyan A. Ju., Kazaryan P. A., Shirvanyan Ju. A., Galoyan A. A. The peculiarities of the changes of hematomorphological indices of the blood of the cows with leucosis under the influence (in vitro) of Proline-rich Polypepeptide (PRP)//Dokladi NAS RA-2005-V.105-N 1-P.86-92.
6. Shishkov V. P., Burba L. G.. Leucosis of agricultural animals//M. “Agropromizdat”-1988
7. Galoyan A. A. Neurochemistry of brain neuroendocrine immune system: Signal molecules. Biochemical and Molecular-Biological Aspects of the Brain Immune System (Encyclopedia Armenica)-Yerevan-2001-P.22-34.
8. Galoyan, A. A.. Brain Neurosecretory cytokines: Immune Response and Neuronal Survival//New York, Kluwer Academic-2003.
9. Galoyan, A. A., Aprikyan, V. S. A new hypothalamic polypeptide is a regulator of myelopoiesis//Neurochem Res.-2002-Vol.27(4)-P.305-312.
10. RU 2188655, A61K35/26, 2002.09.10.

1. (canceled)

2. (canceled)

3. (canceled)

4. A method of treating, or for prophylaxis of, cattle leucosis, the method comprising administering at least one dose of a medicament to an animal, the medicament comprising a peptide having the amino acid sequence Ala-Gly-Ala-Pro-Glu-Pro-Ala-Glu-Pro-Ala-Gln-Pro-Gly-Val-Tyr.

5. A method according to claim 4 wherein the concentration of the peptide in the medicament is at least 1.57×10⁻⁴ g/ml.

6. A method according to claim 5 wherein the at least one dose is administered in a dosage of at least 4×10⁻³ ml of medicament per kg of live animal weight.

7. A method according to claim 6 wherein the at least one dose is administered at least twice in a 48 hour period.

8. A method according to claim 4 wherein the at least one dose is administered in a dosage of at least 6.3×10⁻⁷ grams of peptide per kg of live animal weight.

9. A method according to claim 8 wherein the at least one dose is administer at least twice in a 48 hour period.

10. A method according to claim 4, further comprising monitoring the blood of the animal.

11. A medicament for treating, or for prophylaxis of, cattle leucosis, the medicament comprising a peptide having the amino acid sequence Ala-Gly-Ala-Pro-Glu-Pro-Ala-Glu-Pro-Ala-Gln-Pro-Gly-Val-Tyr.

12. A medicament according to claim 11 wherein the concentration of the peptide in the medicament is at least 1.57×10⁻⁴ g/ml.
